(19) United States
(12) Patent Application Publication
(10) Pub. No.: US 2013/0288225 A1
(43) Pub. Date: Oct. 31, 2013

(54) METHOD FOR SEPARATING WHOLE BLOOD

(71) Applicants: Noha Elmouelhi, Randolph, NJ (US); Sriram Natarajan, Hillsborough, NJ (US); Kevin Cooper, Flemington, NJ (US); Murty Vyakarnam, Bridgewater, NJ (US); Chee Tiong Lim, Singapore (SG); Emma Kim Luong-Van, Singapore (SG); Isabel Rodriguez, Singapore (SG); Hong Yee Low, Botannia (SG); Audrey Yoke Yee Ho, Crescent (SG)

(72) Inventors: Noha Elmouelhi, Randolph, NJ (US); Sriram Natarajan, Hillsborough, NJ (US); Kevin Cooper, Flemington, NJ (US); Murty Vyakarnam, Bridgewater, NJ (US); Chee Tiong Lim, Singapore (SG); Emma Kim Luong-Van, Singapore (SG); Isabel Rodriguez, Singapore (SG); Hong Yee Low, Botannia (SG); Audrey Yoke Yee Ho, Crescent (SG)

(73) Assignees: AGENCY FOR SCIENCE TECHNOLOGY AND RESEARCH, Connexis (SG); ADVANCED TECHNOLOGIES & REGENERATIVE MEDICINE, LLC, Raynham, MA (US)

(21) Appl. No.: 13/458,825

(22) Filed: Apr. 27, 2012

(51) Int. Cl. C12N 5/078

(52) U.S. Cl. 435/2

(57) ABSTRACT

A method of separating blood by providing a horizontal substrate having a plurality of oxygen plasma-treated polypropylene pillars extending from the surface of a polypropylene film, depositing a whole blood sample on an upper surface of the substrate, collecting red blood cells on the upper surface of the pillars and permitting remaining components of said whole blood sample to flow downward and through the pillars.

### FIELD OF THE INVENTION

The present invention relates to polymeric substrates which can effect separation of red blood cells from whole blood.

### BACKGROUND OF THE INVENTION

There is an ongoing need for substrates having improved blood separation activity. Such structures can be suited for use in various applications, such as medical applications in which separation of red blood cells from the remaining whole blood components is desired.

The ability to measure a wide variety of physiologically active compounds, both naturally occurring and synthetic, has become of increasing importance, as an adjunct to both diagnosis and therapy. While for the most part such assays have required clinical laboratory determinations, there is an increasing awareness of the importance of being able to conduct assay determinations in a physician's office or in the home.

In clinical assays, the separation of serum or plasma from whole blood is extremely important since it is often difficult to conduct the analysis of dissolved blood components without interference from the red blood cells. Serum or plasma is conventionally separated from erythrocytes by centrifuging. Centrifugation, however, causes other problems because one must then separate the supernatant from the blood cake. Moreover, this method is not available for use in home or office diagnostic assays.

U.S. Pat. No. 4,464,254 discloses a process for separating plasma or serum from whole blood using a filter of glass fibers. The glass fibers used have an average diameter of 0.2 to 5 microns, and a density of about 0.1 to 0.5 g/cm. Whole blood is placed onto a layer of glass fibers, and plasma is generated by retardation of flow of the cells. Plasma is collected at the other side of the glass fibers.

Another approach to separating red blood cells from whole blood is shown in U.S. Pat. No. 4,753,776. In this patent, capillary action is used to pull whole blood through a glass microfiber filter by retarding the flow of the cells.

U.S. Pat. No. 4,256,693 discloses a multilayered integral chemical analysis element for blood comprising a filter layer capable of removing formed components from the blood. The filter layer may be made of at least one component selected from paper, nonwoven fabric, sheet-like filter material composed of powders or fibers such as man-made fibers or glass fibers, and membrane filters having suitable pore sizes. The filter layer separates the formed components of the blood at one time, or successively, such as in the order of leukocytes, erythrocytes, and platelets.

JP 2004-170935 discloses a microbiological chip comprising a functional substrate equipped with a tiny columnar protrusion group having a first base substance made of an organic polymer and a group of tiny columnar protrusions made of an organic polymer extending from the base substance characterized by the equivalent diameter of the tiny columnar protrusion group being 10 nm to 500 μm, the height being 50 nm to 5,000 μm, and the ratio (H/D) of the equivalent diameter (D) with respect to the height (H) of the tiny columnar protrusion group being 4 or more. The microbiological chip is illustrated in FIGS. 7 and 8, which provides a horizontal flow path through the protrusion group. Flow is effected by electrophoresis.

U.S. Patent Publication 2007/0227967 discloses a filter for separating blood cells, which comprises: a substrate; and at least one water-insoluble substance fixed to the substrate, wherein the at least one water-insoluble substance has an equivalent circle diameter of 4 μm or less and a height equal to or larger than the equivalent circle diameter, wherein blood cells are separated by substantially being captured with the at least one water-insoluble substance; and a blood filtration instrument and a blood analytical device, using the filter for filtration of blood and body fluid.

A need exists for further improved systems and methods for collecting blood components in a way that lends itself to use in online blood collection environments, where high yields of critically needed cellular blood components, like plasma, red blood cells, and platelets, can be realized in reasonably short processing times. Furthermore, a need exists for a passive system to separate red blood cells from whole blood that can be easily incorporated into devices (e.g. diagnostic devices), which has a large surface area for separation.

The present inventors have discovered an integrally formed substrate, made from a single synthetic polymer, which acts to separate red blood cells from remaining whole blood components simply and with high surface area.

### SUMMARY OF THE INVENTION

The present invention relates to a method of separating blood, comprising providing a horizontal substrate comprising a plurality of oxygen plasma-treated polypropylene pillars extending from the surface of a polypropylene film, depositing a whole blood sample on an upper surface of said substrate, collecting red blood cells from said whole blood sample deposited on said upper surface on an upper surface of said polypropylene pillars, permitting remaining components of said whole blood sample to flow downward and through said polypropylene pillars, and collecting said red blood cells and/or said remaining components of said whole blood sample.

### BRIEF DESCRIPTION OF THE DRAWINGS

FIG. 1 depicts a scanning electron micrograph of red blood cells collected on top of the polypropylene pillars according to the present invention.

### DETAILED DESCRIPTION

The invention is directed to a blood separation film having integral, high aspect ratio (HAR), length to diameter polypropylene (PP) sub-micron to micron-sized densely packed pillared surface features on the film, that when oxygen plasma-treated, will effect separation of red blood cells from the remaining components of whole blood, by collecting red blood cells on the top surfaces of the pillars, while the remaining components penetrate down to the surface of the underlying substrate.

In one embodiment, the surface of said polypropylene film is perforated and said remaining components of said whole blood sample are collected below the film.

In another embodiment, the invention is directed to a blood separating medical device, comprising a blood separation film having a plurality of oxygen plasma-treated polypropylene pillars extending from the surface thereof.

In one embodiment, the pillars have an average diameter ranging from 0.2 to 5 microns, preferably from about 0.8 μm to about 3 μm, and aspect ratios (length/diameter) of from about 0.5 to about 40, preferably from about 2 to about 25. In a particularly preferred embodiment, the pillars have a diameter of about 1 μm and a height of about 20 μm. Advantageously, the pillars are densely packed so as to preclude red blood cells from passing through, having spacing between them of less than about 3 μm, preferably less than about 2 μm, and in a particularly preferred embodiment, less than about 1 μm.

The surfaces of the blood separation films/pillars are treated by oxygen plasma treatment using a microwave plasma processor (100 W, 30 seconds). Oxygen plasma treatment results in the polypropylene film and pillars having a higher oxygen content on the surface thereof as compared to non-oxygen plasma-treated polypropylene film and pillars. Depending on the severity of the treatment, the polypropylene film and pillars can have an oxygen content of about 21%.

It has been determined that the oxygen-plasma treated films and pillars result in a blood separation substrate having static water contact angles of less than about 15°, such that the substrate demonstrates super-hydrophilicity, even though polypropylene is well-known to be highly hydrophobic.

In yet still another embodiment, the blood separation substrate surface is substantially planar and the pillars are within about ±45 degrees of normal to the planar surface, more preferably within about ±30 degrees of normal to the planar surface.

The blood separation substrate is at least partially formed by a process selected from nano- or micro-molding using a template, polymer self-assembly, lithography, and etching. For example, a method of forming the blood separation substrate comprises a) providing a specific solvent-dissolvable mold including indentations; b) providing a polypropylene film to the mold under conditions sufficient to permit filling the indentations of the mold by the polypropylene; c) treating the mold and polypropylene of step b) to an extent sufficient to substantially solidify the polypropylene; d) exposing the mold and polypropylene the specific solvent (selected to dissolve the mold but not the polypropylene) under mold-dissolving conditions to provide a pillared substrate; and e) oxygen plasma-treating the pillared substrate.

The invention is further explained in the description that follows with reference to the drawings illustrating, by way of non-limiting examples, various embodiments of the invention.

### Example 1

This example shows that polypropylene densely-packed surface structures of high aspect ratio can be oxygen plasma-treated to separate red blood cells from a whole blood sample. Polypropylene pillars of diameter 1 micron and height 20 micron were fabricated using a polycarbonate membrane as a mold and an imprinting process as follows:
A commercial track-etched polycarbonate membrane was obtained from Millipore Corporation of Billerica, Mass., USA of having pores of 1 micron diameter and a circular diameter of 2.5 cm, with a thickness of 20 micron.
The membrane was used as a template to imprint a solvent-resistant polypropylene polymer film of 300 micron thickness, obtained from Ethicon, Inc. of Somerville, N.J., USA. The polypropylene film was pressed into the polycarbonate membrane template under high temperature and pressures (180° C., 600 kPa (6 bar)) for 20 minutes, melting the polypropylene.
The polypropylene polymer and the membrane are cooled to 60° C. before removal of pressure, after which the polymer structures are de-molded and released by dissolving the membrane in dichloromethane.
Films were oxygen plasma-treated using a microwave plasma processor at 100 W for 30 seconds.

The porous solvent-dissolvable polycarbonate material which acts as a template for the pillar-like pillars of the product can be substituted by another solvent-dissolvable porous polymeric material. Alternately, a strippable mold such as anodized aluminum oxide can be substituted to provide the pillar-like cylindrical pillars of the final product, without the need for exposure to a chemical solvent. Polyimide film (sold under the tradename KAPTON™ by E.I. du Pont de Nemours and Company, Wilmington, Del.) was used as a capping means or shield to protect polymer surfaces from directly contacting surfaces such as metal. Other suitable substantially chemically inert materials which can also be provided as a film or other layer for this purpose include polytetrafluoroethylene (sold under the tradename TEFLON™ by E.I. du Pont de Nemours and Company, Wilmington, Del.). Advantageously, these materials are not reactive with the polycarbonate solvent-dissolvable mold or template material and can be readily removed or peeled therefrom once compression is completed.

Static water contact angle measurements, herein referred to as contact angle measurements, were conducted using a sessile drop method. A Rame-Hart contact angle goniometer with Drop Image software was used. Plasma treatment was done immediately before contact angle measurement. Two microliter drops of de-ionized water were placed on the surface for measurement, and 5 measurements were taken for each surface. The mean contact angle is reported in Table 1, below.

TABLE 1

PP Water Contact Angles

| | Untreated | Treated |
|---|---|---|
| Flat | 101° | 59° |
| Pillars | 148° | 14° |

The contact angle of the untreated pillared structures is higher than the corresponding flat film (148° vs 101°), implying their greater hydrophobicity or non-wettability. Oxygen plasma treatment greatly reduces the contact angle for water on these surfaces, as shown in Table 1, resulting in wettable surfaces (and greater hydrophilicity).

Surface elemental analysis was conducted on flat films with results shown in Table 2, below. The oxygen plasma treatment results in a higher oxygen content on the film (20.9% versus 4.3%).

TABLE 2

| Element | Untreated PP | Plasma-treated PP |
|---|---|---|
| C | 95.7% | 79.1% |
| O | 4.3% | 20.9% |

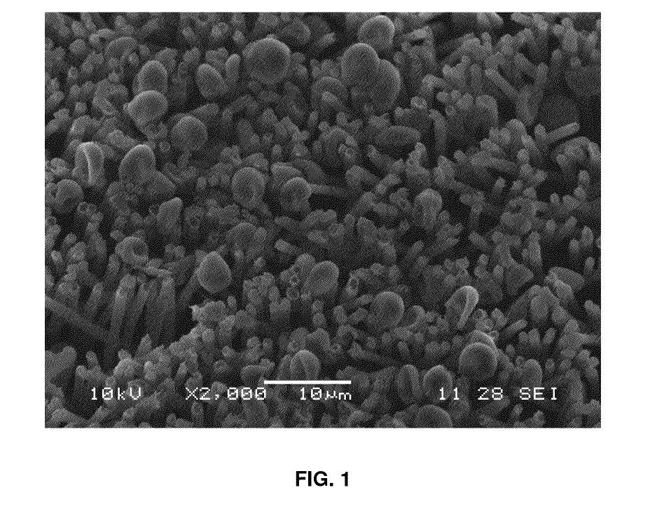

Rabbit blood was collected into EDTA tubes (BD Biosciences). Polypropylene films according to the invention were placed in contact with EDTA anti-coagulated blood in a microcentrifuge tube and incubated for 90 minutes in a 37° C. water bath. The films were rinsed three times with phosphate buffered saline (PBS) and fixed with 2.5% gluteraldehyde for 2 hours at 4° C., followed by an ethanol dehydration series of 0, 25, 50, 75 and 100% ethanol in deionized water. Samples were coated with gold for 20 seconds before SEM imaging. FIG. 1 depicts an SEM of the structures after exposure to whole blood. Red blood cells can be seen on the top of the structures.

All patents, test procedures, and other documents cited herein, including priority documents, are fully incorporated by reference to the extent such disclosure is not inconsistent and for all jurisdictions in which such incorporation is permitted.

When numerical lower limits and numerical upper limits are listed herein, ranges from any lower limit to any upper limit are contemplated.

The invention being thus described, it will be apparent that the same may be varied in many ways. Such variations are not to be regarded as a departure from the spirit and scope of the invention, and all such modifications as would be obvious to one skilled in the art are intended to be included within the scope of the following claims.

1. A method of separating components of whole blood, comprising:
providing a horizontal substrate comprising a plurality of oxygen plasma-treated polypropylene pillars extending from the surface of a polypropylene film;
depositing a whole blood sample on an upper surface of said pillars, such that red blood cells from said whole blood sample are retained on said upper surface while remaining components of said whole blood sample flow downward between said polypropylene pillars to the surface of said polypropylene film; and
collecting either said red blood cells or said remaining components of said whole blood sample.

2. The method of claim 1, wherein the pillars are of a diameter from about 0.8 μm to about 3 μm, have aspect ratios from about 0.5 to about 40, and are spaced apart by less than about 3 μm.

3. The method of claim 1, wherein said substrate has a static water contact angle of less than about 15°.

4. The method of claim 1, wherein the polypropylene film and pillars have a higher oxygen content on the surface thereof than non-oxygen plasma treated polypropylene film and pillars.

5. The method of claim 1, wherein the polypropylene film and pillars have an oxygen content of about 21%.

6. The method of claim 2, wherein the pillars have a diameter of about 1 μm, a height of about 20 μm, and are spaced apart by less than about 2 μm.

7. The method of claim 1, wherein said polypropylene pillars are substantially cylindrical.

8. The method of claim 1, further comprising collecting the remainder of said whole blood sample.

9. The method of claim 1, wherein the surface of said polypropylene film is perforated and said remaining components of said whole blood sample are collected below the film.
